(19) United States
(12) Patent Application Publication
(10) Pub. No.: US 2026/0269073 A1
(43) Pub. Date: Sep. 10, 2026

(54) FUNCTIONAL BODY SYSTEM ASSESSMENT AND DYSFUNCTION PREDICTION USING SYMPTOM AND BIOMARKER DATA

(71) Applicant: Bioinsights, Lutz, FL (US)

(72) Inventors: Travis Lamar Bond, Lutz, FL (US); Erin Bolton Coletti, Belleair, FL (US)

(73) Assignee: Bioinsights, Lutz, FL (US)

(21) Appl. No.: 19/467,680

(22) Filed: Feb. 2, 2026

Related U.S. Application Data
(60) Provisional application No. 63/753,343, filed on Feb. 3, 2025.

(51) Int. Cl. G16H 50/30; G16H 50/20

(52) U.S. Cl. CPC G16H 50/30; G16H 50/20

(57) ABSTRACT

Various techniques involve accessing stored data indicating health conditions of a plurality of subjects and a request for information about a particular subject. It can be determined whether the stored data includes at least one of: (i) symptom data, (ii) first biomarker data, or (iii) second biomarker data. A first set of weights can be applied to determine a composite score based on the stored data including all of the symptom data, the first biomarker data, and the second biomarker data. A second set of weights can be applied to determine the composite score based on a the stored data not including at least one of the symptom data, the first biomarker data, or the second biomarker data. A probability of the particular subject having a functional condition can be estimated based on the composite score.

### CROSS-REFERENCE TO RELATED APPLICATIONS

This application claims the benefit of U.S. Provisional Patent Application No. 63/753,343 filed on Feb. 3, 2025. The entire disclosure of the aforementioned application is incorporated by reference herein in its entirety for all purposes.

### BACKGROUND

Computer systems can be used to identify dysfunctions that may cause an imbalance or disease in a subject. For example, a computer system can be used to access medical records and/or other data associated with a subject to facilitate recommending a clinical treatment for restoring balance, health, and/or well-being of the subject.

### BRIEF SUMMARY

In some embodiments, a computer-implemented method includes accessing stored data indicating health conditions of a plurality of subjects and a request for information about a particular subject. The method also includes determining whether the stored data includes at least one of: (i) symptom data, (ii) first biomarker data, or (iii) second biomarker data. The method also includes applying a first set of weights to determine a composite score based on the stored data including all of the symptom data, the first biomarker data, and the second biomarker data. The method also includes applying a second set of weights to determine the composite score based on the stored data not including at least one of the symptom data, the first biomarker data, or the second biomarker data. The method also includes estimating a probability of the particular subject having a functional condition based on the composite score.

In one embodiment, a system includes one or more data processors and non-transitory computer-readable storage medium storing instructions which, when executed on the one or more data processors, cause the one or more data processors to perform part or all of one or more methods disclosed herein.

In one embodiment, a computer-program product tangibly embodied in a non-transitory machine-readable storage medium that stores instructions configured to cause one or more data processors to perform part or all of one or more methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates a flow chart of an example process that predicts body dysfunctions and their severity using various combinations of symptom data and/or biomarker data.

FIG. 2 illustrates a system diagram showing an example dysfunction prediction system that predicts body dysfunctions using a combination of symptom data and biomarker data.

FIG. 3 illustrates a diagram of an example user interface showing an example subject profile summary.

FIG. 4 illustrates a diagram of an example user interface showing an example dysfunction prediction.

FIG. 5 illustrates a diagram of a distributed system for dysfunction prediction.

FIG. 6 illustrates an example computer system that may be used to predict body dysfunctions using a combination of symptom data and biomarker data.

### DETAILED DESCRIPTION

Example systems and methods are described for predicting body dysfunctions using a combination of subjective symptom data and objective biomarker data. In various embodiments, the example dysfunction prediction system that predicts body dysfunctions using a combination of symptom data and biomarker data is implemented using non-transitory computer-readable storage media to store instructions which, when executed by one or more processors of a computer system, cause display of the user interface and processing of the received input to predict body dysfunctions using a combination of symptom data and biomarker data.

A description of systems and methods for functional body system assessment and dysfunction prediction using symptom and biomarker data is provided in the following sections:
FBS ASSESSMENT AND FUNCTIONAL CONDITION DETECTION
ANALYZING AND INTEGRATING DATA FROM MULTIPLE DATA SOURCES
DYNAMICALLY COMPUTING BURDEN SCORES FOR SYMPTOM AND BIOMARKER DATA METRICS OF A SPECIFIC FBS SUBSYSTEM
DYNAMIC WEIGHTING OF BURDEN SCORES BASED ON DATA AVAILABILITY TO GENERATE A COMBINED SCORE
USING DYNAMICALLY COMPUTED AND WEIGHTED SCORES TO PREDICT FUNCTIONAL CONDITIONS
AUTOMATED GENERATION OF PERSONALIZED CLINICAL EVALUATIONS, TREATMENT PLANS, AND LAB TESTING RECOMMENDATIONS
COMPUTER SYSTEM ARCHITECTURE

The steps described in individual sections may be started or completed in any order that supplies the information used as the steps are carried out. The functionality in separate sections may be started or completed in any order that supplies the information used as the functionality is carried out. Any step or item of functionality may be performed by a personal computer system, a cloud computer system, a local computer system, a remote computer system, a single computer system, a distributed computer system, or any other computer system that provides the processing, storage and connectivity resources used to carry out the step or item of functionality.

### Functional Body System (FBS) Assessment and Functional Condition Detection

Functional medicine practitioners typically employ a holistic approach to health by considering subjective symptoms and/or objective biomarker data. However, in some scenarios, providing a robust clinical evaluation or dysfunction diagnosis according to this holistic approach may be challenging due to reliance on a combination of subjective symptom data (if available) and/or objective marker data (if available) across different subject profiles. Furthermore, in some scenarios, updated medical information relevant to a particular FBS or functional condition (i.e., dysfunction) may become available after an assessment is performed. For example, if a specific biomarker is recently identified (e.g., in a clinical research trial, etc.) as being a relatively strong indicator for a specific functional condition, the practitioner may want to adjust their previous FBS assessments and/or functional condition predictions to more accurately evaluate whether or not a previously assessed subject have a particular dysfunction in light of the updated medical information. However, it may be technically challenging to identify every potentially affected subject accurately and efficiently every time such updated information becomes available, including subjects who were incorrectly assessed as having or as not having that particular dysfunction.

The present disclosure includes example systems and methods for dynamically integrating various types of available subject data (e.g., symptom data, biomarker data, etc.) and/or data weights (e.g., biomarker relevancy to a specific condition, etc.) in an efficient and consistent manner to enable practitioners and/or other system users to better predict and/or evaluate various functional conditions.

In some aspects, examples described herein include estimating the probability of a subject having one or more specific dysfunctions in a flexible or customizable manner depending on currently available subject data. For example, a system may use a first set of weights to weigh biomarker data used to predict dysfunction probability if symptom data (e.g., questionnaire results) is also available. Whereas, if the symptom data is not available for a particular subject, the system may instead use a second (different) set of weights to weigh the biomarker data when predicting dysfunction probability.

In some aspects, examples described herein include dynamically computing burden scores, e.g., scores that characterize the severity of a symptom or biomarker data metric with respect to a potential dysfunction or functional condition prediction. In some aspects, examples described herein include integrating biomarker rule sets, e.g., to emphasize certain biomarkers deemed as strong indicators of a particular dysfunction. In some aspects, examples described herein include assigning severity flags, e.g., graphical user interface (GUI) indicators of severity classifications such as mild, moderate, severe, etc., corresponding to severity of a symptom or dysfunction. In some aspects, examples described herein involve interpreting and/or displaying data analysis results, e.g., predictive dysfunction analysis results may be presented in a natural language format, etc. In one example, a system may determine a likelihood of subjective and/or objective observable dysfunctions in a body of a subject based on currently available data, suggest additional lab tests or symptom questions to improve a robustness or accuracy of its prediction, update its assessment dynamically when additional relevant data becomes available, and/or provide treatment recommendations based on its assessment.

In the field of functional medicine, a functional condition may refer to a state where bodily systems of an individual are not operating optimally, which may sometimes result in various symptoms, without necessarily being associated with a specific identifiable structural abnormality or disease. Thus, example systems described herein may facilitate better understanding and addressing of underlying dysfunctions associated with interconnected systems of a body, in addition to or instead of identifying or addressing specific diseases.

By way of example, some functional conditions may be characterized by persistent and/or troublesome symptoms that affect daily functioning or quality of life, even though standard medical tests do not necessarily associate these symptoms with a specific structural cause. A non-exhaustive list of examples of such functional conditions includes irritable bowel syndrome (IBS), fibromyalgia, and/or functional neurological disorders, among other examples. For example, one or more of these functional conditions may be caused by complex interplay between genetics, environment, lifestyle, and/or psychosocial factors, in addition to or instead of a specific structural abnormality or disease.

Examples described herein may involve identifying and addressing such underlying dysfunctions through a personalized, system-oriented, and/or other robust data analysis process. For instance, an example system may generate recommendations and/or treatment plans that facilitate restoring balance and/or improving health outcomes by accounting for genetic, environmental, lifestyle influences, and/or other relevant data (e.g., biomarker data, lab test results, etc.) associated with a particular subject. Thus, in some aspects, the present approach includes examples aligned with an evolving understanding of health and disease as being part of a continuum, e.g., where function or evaluation can move both forward and backward. In some aspects, examples described herein may facilitate developing more effective, individualized treatment plans by focusing on dynamic processes that may cause a dysfunction, rather than merely labeling or fitting subject symptoms/biomarker data as corresponding to certain diseases. In some aspects, the present approach accounts for scenarios where some symptoms may result from complex interactions within the systems of a body and/or with the external environment that are not necessarily associated with a particular disease or structural abnormality.

Analyzing and Integrating Data from Multiple Data Sources

Example systems and methods described herein include a computing platform configured to collect and analyze data relevant to assessing status of a particular FBS of a subject from multiple data sources. A non-exhaustive list of example data sources includes symptoms data (e.g., questionnaire responses provided by subject), lab results (e.g., biomarker measurements or other lab panel results), and/or other data sources (e.g., medical diagnosis, meeting notes, conversation transcripts, prescription history, subject health goals, subject complaints, and/or any other relevant data input from a user).

In one embodiment, an example system includes a functional condition database storing information about one or more functional conditions to be evaluated. For example, a team of experts may use a user interface of the system to input a list of common dysfunctions (e.g., upper gastrointestinal, vitamin need, adrenal, etc.) in the functional condition database. The system may also include a symptom repository storing questions about symptoms (e.g., genetic information, medical history, sleep patterns, fatigue, etc.) that may potentially indicate one or more functional conditions. For instance, the system may store a mapping that maps a given symptom associated with a question in the symptom data repository to one or more functional conditions in the functional condition database. In some examples, the symptom repository may include questions about symptom frequency (e.g., daily, monthly, weekly, etc.). The system may also include a biomarker data repository (e.g., lab test results, medical panels, etc.) that stores data identifying objectively measurable biomarkers (e.g., gene sequences, etc.) associated with one or more functional conditions. In some examples, the system may store or otherwise access, in association with the biomarker data repository, information indicating an optimal and/or normal reference range of values for each biomarker. In some examples, the system may store information that identifies a certain biomarker (e.g., ‘HbA1c’ gene) as a strong indicator of a certain functional condition (e.g., diabetes). Other database structures, configurations, and/or stored data are possible as well without departing from the scope of the present disclosure.

In one embodiment, an example system includes a user interface configured to receive a request from a user (e.g., clinic practitioner) to generate and/or send an invitation (e.g., via electronic mail, etc.) to a subject of the user. For instance, the system may use an application programming interface (API) to interface with an e-mail application to send an invitation to the subject. In one example, the invitation may include a link to a comprehensive intake form that gathers information (e.g., based on questions in a questionnaire repository) about symptoms and other relevant subjective data from the subject (e.g., demographics, medical history, genetic information, family history, detailed system review categorized by FBS, etc. In some examples, the user interface of the system may include an option for the practitioner to filter the questions in the questionnaire, e.g., to select questions about symptoms associated with a particular potential functional condition that the practitioner wants to analyze. In some examples, the user interface may generate a report that summarizes responses input by the subject and display the report to the practitioner user for further confirmation of the intake questionnaire responses and/or for further discussion with the subject about the responses to improve clarity and/or accuracy of the responses.

In some examples, the system may generate and/or display (in the user interface) a subject assessment report displaying a burden score computed for each FBS of the set of FBSs associated with the symptoms indicated in the questionnaire responses. The burden score may be a score that indicates an extent or probability that the subject may have a certain dysfunction (e.g., cellular dysfunction) associated with each respective FBS. In some examples, the system may generate and/or display (via the user interface) one or more lab test recommendations associated with one or more predicted dysfunctions (e.g., predicted based on the symptom data with or without relevant lab test results being available). For example, the system may execute one or more algorithms to identify one or more specific laboratory tests that could help assess various FBS subsystems (e.g., subsystems of interest, or a comprehensive list of FBS subsystems). For example, the system may update the user interface to display one or more orders for lab tests to be ordered for the subject. In some examples, upon receiving lab test results (e.g., either from new orders or existing subject records), the system may be configured to evaluate and/or classify the various test results to update any relevant FBS burden scores and/or dysfunction predictions accordingly.

### Dynamically Computing Burden Scores for Symptom and Biomarker Data Metrics of a Specific FBS Subsystem

In some aspects, examples described herein include computing various scores to quantify the severity of subjective and/or objective data measurements associated with each FBS subsystem. In an example embodiment, an example system is configured to compute a symptom burden score (‘S’), a biomarker burden score (‘B’), and a biomarker rule set burden score (‘R’).

The symptom burden score may include any metric that quantifies severity of subject-reported symptoms associated with each FBS subsystem. For instance, the system may assign a certain number of points for each symptom reported by the subject that is relevant to a particular FBS subsystem.

In one example, the system may assign points based on symptom frequency (e.g., monthly symptoms assigned 1 point, weekly symptoms assigned 2 points, daily symptoms assigned 3 points, etc.). In another example, a symptom may be assigned a different number of points depending on the question configuration in the symptoms database (e.g., either 1 or 2 points, only 1 point, etc.). In yet another example, the system may be configured to receive input from a user (e.g., practitioner, etc.) to assign or select a custom or specific weight/number of points as corresponding to a particular symptom or questionnaire response. Alternatively or additionally, in examples, the system can be configured to assign a first weight and/or score for a symptom if it is being considered as an indicator of a first functional condition and a different second weight or score if the same symptom is being considered as an indicator of a second functional condition.

Continuing with this example, the system may be configured to calculate a total symptom score (‘S_{T}’) for all the symptoms of a particular FBS subsystem reported by the subject. For example, S_{T} may be computed as a sum of the points (e.g., 1, 2, 3, etc.) of all positive symptoms reported by the patent that are associated with the particular FBS subsystem. The system may also determine a maximum possible score (‘M’), where M is a sum of the maximum number of points possible if all the symptoms associated with the FBS subsystem were reported at the highest possible symptom frequency or point value (i.e., maximum points per symptom). In this example, the system may then determine a System Severity Percentage or symptom burden score (‘S’) using the equation below:

$$System Severity Percentage ( S ) = ( {S}_{T} / M )$$

In some examples, the system is configured to interpret the severity of subject-reported symptoms indicated by the system burden score(S) using one or more value ranges. For example, an S value of between 0%-10.99% may be interpreted, e.g., by displaying a particular graphic indicator such as a green colored indicator in the user interface, as an optimal symptom severity. As another example, a range of 11%-16.99% may indicate a mild symptom severity (e.g., yellow colored indicator), a range of 17%-32.99% may be interpreted as moderate severity (e.g., orange colored indicator), and a range of greater than or equal to 33% may be interpreted as high severity (e.g., red colored indicator), etc. It should understood that these range values are only provided for the sake of example. The system can alternatively be configured to assign different ranges as corresponding to different severity levels.

The biomarker burden score (‘B’) may include any score that assesses a degree of dysfunction associated with a particular FBS subsystem based on lab panel results. In an example, a system is configured to determine a total number of biomarkers measured (‘Y’), which may include biomarkers in composite tests (e.g., biomarker tests associated with more than one FBS subsystem). The system may also compute a number of biomarkers (‘A’) outside a normal reference range, i.e., abnormal. Abnormal biomarkers (A) are biomarkers that may be outside a normal reference range which may indicate significant physiological disturbances. The system may also compute a number of biomarkers (‘B_{N}’) within the normal reference range but outside an optimal range, i.e., suboptimal. Sub-optimal biomarkers (‘B_{N}’) may be biomarker measurements within the normal reference range but outside an optimal reference range, which may suggest potential early dysfunction or risk. For instance, ‘optimal’ values may be values considered as ‘ideal’ and thus may be assigned a low or no weight toward dysfunction (e.g., cellular dysfunction). Thus, in one example, the system may assign a weight of 4 for abnormal biomarkers (A), a weight of 2 for normal biomarkers (B_{N}), and a weight of 0 for optimal biomarkers. The following equation describes an example computation of the biomarker burden score (‘B’).

$$Biomaker Burden Score ( B ) = ( 4 A + 2 {B}_{N} + 1 ) / 3 Y$$

In this example, multiplying Y by 3 may result in the maximum possible score (e.g., normalizing the burden score to a percentage). The ‘+1’ offset may be included in this equation to ensure that there is no zero value biomarker weight in overall calculation. It is noted that other weights, values, and/or normalization computations are possible for computing the biomarker burden score B.

In some examples, the system is configured to use different weight values for each abnormal biomarker (A) based on its degree of abnormality (e.g., instead of or in addition to the single weight of 4 for all abnormal biomarkers). For instance, weighting biomarkers according to their degree of abnormality may allow for a more nuanced assessment. Thus, in these examples, the system may improve precision of dysfunction prediction which may result in better informed clinical decisions.

The biomarker rule set burden score (‘R’) may include any score configured to capture specific biomarker patterns or metrics deemed as biomarkers that strongly indicate a particular dysfunction within an FBS subsystem. For example, a user (e.g., clinician) and/or medical expert may designate one or more particular biomarkers (e.g., in a biomarker database of the system) as a biomarker rule set (i.e., biomarkers that should be afforded additional weight, etc.). In an example, a system may compute R to incorporate weights for abnormal and/or sub-optimal biomarkers in a similar manner as that used to compute B, for example:

$$R = ( 4 {A}_{r} + 2 {B}_{r} ) / 3 {Z}_{max}$$

In this example, Aᵣ may correspond to a number of rule-set abnormal biomarkers (e.g., outside the normal reference range), Bᵣ may correspond to a number of rule-set biomarkers that are sub-optimal (e.g., within the normal range but outside the optimal range), and Zₘₐₓ may correspond to a total number of biomarkers in the rule set. Thus, in this example, The numerator (4Aᵣ+2Bᵣ) may assigns a weight of 4 to abnormal biomarkers and a weight of 2 to sub-optimal biomarkers within the rule set, e.g., reflecting their relative clinical importance. Optimal markers, in this example, may be considered (e.g., ideal) biomarker values that have a weight value of zero. Further, in this example, the denominator (3Zmax) may represent the maximum possible weighted score if all biomarkers in the rule set were abnormal (e.g., 3 times the total number of biomarkers).

In some examples, the system is configured to provide customizable biomarker weights (e.g., instead of the fixed values of 4 and 2 used in the two examples of R and B above) for one or more FBS subsystems.

In some examples, a system may compute and/or update the various burden scores (e.g., S, B, R) described above dynamically as data (e.g., symptom data, biomarker data, etc.) is input to the system. For example, when a subject submits new or updated questionnaire responses, the system may automatically compute updated S burden scores for each FBS subsystem that account for the new symptom question responses. As another example, if lab test provider submits new lab panel results, the system may update B and R scores accordingly to account for the new biomarker data received for a particular subject. Thus, in this example, a user (e.g., practitioner) may view the most up-to-date severity assessments and/or functional condition predictions at any time by accessing a particular subject profile page via the platform.

### Dynamic Weighting of Burden Scores Based on Data Availability to Generate a Combined Score

In some scenarios, the system may generate a combined burden score for the various types of data (e.g., S, B, R, etc.) based on data availability. For example, if the system has both symptom data and biomarker data, the system may compute a weighted average of S and B, and so on. The weights used for each combination may be different and customizable so that the combined score is more indicative of the severity in light of the available data.

In some embodiments, an example system is configured to determine a combined FBS score ('S+B′) based on a weighted average of the symptom burden score(S) and the biomarker burden score (B) of the FBS or FBS subsystem. For example, the system may be configured to display (e.g., via a user interface) the functional status of a subject as a graphical representation of various functional states of various functional body systems and/or FBS subsystems of the subject. In one example, the user interface may depict the severity of potential dysfunctions associated with each FBS or FBS subsystem using a color UI element or other indicator (e.g., green for optimal score, yellow for mild, orange for moderate, red for severe, etc.). To facilitate this, the system may be configured to compute a combined burden score ('S+B′) based on a combination of the symptom burden score(S) and the biomarker burden score (B) of each FBS subsystem. For example, the system may combine both the symptom and biomarker scores by computing a weighted average. In an example, the system uses a weight mapping to assign specific weights for each of the FBS subsystems, as illustrated by exemplary Table 1 below.

TABLE 1
Example Weights for averaging symptom and biomarker burden scores.

| master_fbs_name | fbs_subsystem | symptom_weight | biomarker_weight |
|---|---|---|---|
| Upper | Upper Gastrointestinal | 0.50 | 0.50 |
| Gastrointestinal | System | | |
| Liver and | Liver and Gallbladder | 0.30 | 0.70 |
| Gallbladder | | | |
| Small Intestine | Small Intestine | 0.50 | 0.50 |
| Large Intestine | Large Intestine | 0.50 | 0.50 |
| Mineral Needs | Mineral Needs | 0.40 | 0.60 |
| Essential Fatty Acids | Essential Fatty Acids | 0.40 | 0.60 |
| Blood Sugar | Blood Sugar Regulation | 0.30 | 0.70 |
| Regulation | (Hyper) | | |
| Blood Sugar | Blood Sugar Regulation | 0.30 | 0.70 |
| Regulation | (Hypo) | | |
| Vitamin Need | Vitamin Need | 0.50 | 0.50 |
| Adrenal | Adrenal (Hyper) | 0.30 | 0.70 |
| Adrenal | Adrenal (Hypo) | 0.30 | 0.70 |
| Pituitary | Pituitary (Hyper) | 0.40 | 0.60 |
| Pituitary | Pituitary (Hypo) | 0.40 | 0.60 |
| Thyroid | Thyroid (Hyper) | 0.30 | 0.70 |
| Thyroid | Thyroid (Hypo) | 0.30 | 0.70 |
| Male Only | Male Only | 0.40 | 0.60 |
| Female | Female Only (Hyper) | 0.40 | 0.60 |
| Female | Female Only (Hypo) | 0.40 | 0.60 |
| Cardiovascular | Cardiovascular | 0.30 | 0.70 |
| Kidney | Kidney | 0.35 | 0.65 |
| Bladder | Bladder | 0.40 | 0.60 |
| Immune System | Immune System | 0.50 | 0.50 |
| Cognitive | Cognitive | 0.45 | 0.55 |

In the example of Table 1, the first column (‘master_fbs_name’) shows the functional body systems evaluated by the example system, the second column (‘fbs_subsystem’) shows the names of FBS subsystems associated with each FBS, the third column (‘symptom_weight’) shows symptom weights used when computing a weighted average of the symptom scores(S) and the biomarker scores (R) of each FBS subsystem. For example, some functional body systems (e.g., Upper Gastrointestinal) may have one subsystem (e.g., Upper Gastrointestinal System), while other FBSs (e.g., Blood Sugar Regulation) may have more than one FBS subsystem (e.g., Blood Sugar Regulation (Hyper), Blood Sugar Regulation (Hypo), etc.). In this example, each FBS subsystem may be mapped to a certain combination of symptom scire weight and biomarker score weight. For example, the system may compute a weighted average score for the “Upper Gastrointestinal System” FBS subsystem by using a symptom weight (‘w_{S}’) of 0.5 for the symptom burden score(S) and a biomarker weight (‘w_{B}’) of 0.5 for the biomarker burden score (B) of a particular subject. Thus, for example, the system may impose a constraint on the weighted average computation according to the following equation:

$$Constraints : {w}_{S} + {w}_{B} = 1$$

In some embodiments, an example system is configured to generate a combined score based biomarker data and biomarker rule set data (‘R+B’). For example, in cases where subjects are entered into the system as established subjects, these subjects may not have intake questionnaires, i.e., symptom data was not yet captured. In this example, S=0 and thus the system may be configured to use a different weight mapping for combining R and B. Table 2 below illustrates an example mapping for the weights used to combine R and B in this scenario to compute the weighted average. In this example, biomarker data is used to calculate Severity Scores for FBS Subsystems where data is available. For instance, if lab data is available that only maps to the small intestine, then the system may display severity scores for the small intestine in the subject summary but not for other FBS subsystems where biomarker data is not available.

TABLE 2
Example weight mapping in scenario where symptom data is not available.

| | | | Biomarker_ |
|---|---|---|---|
| master_fbs_name | fbs_subsystem | biomarker_weight | rule_set |
| Upper | Upper Gastrointestinal | 0.50 | 0.50 |
| Gastrointestinal | System | | |
| Liver and | Liver and Gallbladder | 0.50 | 0.50 |
| Gallbladder | | | |
| Small Intestine | Small Intestine | 0.55 | 0.45 |
| Large Intestine | Large Intestine | 0.55 | 0.45 |
| Mineral Needs | Mineral Needs | .60 | 0.40 |
| Essential Fatty Acids | Essential Fatty Acids | 0.65 | 0.35 |
| Blood Sugar | Blood Sugar Regulation | 0.60 | 0.40 |
| Regulation | (Hyper) | | |
| Blood Sugar | Blood Sugar Regulation | 0.60 | 0.40 |
| Regulation | (Hypo) | | |
| Vitamin Need | Vitamin Need | 0.55 | 0.45 |
| Adrenal | Adrenal (Hyper) | 0.55 | 0.45 |
| Adrenal | Adrenal (Hypo) | 0.55 | 0.45 |
| Pituitary | Pituitary (Hyper) | 0.60 | 0.40 |
| Pituitary | Pituitary (Hypo) | 0.60 | 0.40 |
| Thyroid | Thyroid (Hyper) | 0.50 | 0.50 |
| Thyroid | Thyroid (Hypo) | 0.50 | 0.50 |
| Male Only | Male Only | 0.65 | 0.35 |
| Upper | Upper Gastrointestinal | 0.50 | 0.50 |
| Gastrointestinal | System | | |
| Liver and | Liver and Gallbladder | 0.50 | 0.50 |
| Gallbladder | | | |
| Female | Female Only (Hyper) | 0.60 | 0.40 |
| Female | Female Only (Hypo) | 0.60 | 0.40 |
| Cardiovascular | Cardiovascular | 0.60 | 0.40 |
| Kidney | Kidney | 0.65 | 0.35 |
| Bladder | Bladder | 0.60 | 0.40 |
| Immune System | Immune System | 0.55 | 0.45 |
| Cognitive | Cognitive | 0.55 | 0.45 |

In some embodiments, an example system may be configured to generate a combined score ('S+B+R′) based on a weighted average of S, B, and, R. For example, the combined severity score may integrate symptom burden, biomarker burden, and biomarker rule set burden. In this example, the system may be configured to use a different weight mapping such that S, B, and R are weighted according to their importance in each FBS subsystem. The equation below illustrates an example computation of the combined score in this scenario.

$$C = {w}_{S} ( \frac{S}{M} ) + {w}_{B} ( \frac{4 A + 2 B}{3 Y} ) + {w}_{R} ( \frac{4 {A}_{r} + 2 {B}_{r}}{3 {Z}_{max}} )$$

In this example, Aᵣ is the number of rule-set abnormal biomarkers (e.g., outside the normal reference range), Bᵣ is the number of rule-set biomarkers that are sub-optimal (e.g., within the normal range but outside the optimal range), Zₘₐₓ is the total number of biomarkers in the rule set, w_{S} is the weight of symptom burden score, w_{B} is the weight of Biomarker Burden Score, and w_{R} is the weight of Biomarker Rule Set Burden. In this example, the sum of all weights is 1 (e.g., w_{S}+w_{B}+w_{R}=1). In this example, the numerator (4Aᵣ+2Bᵣ) assigns a weight of 4 to abnormal biomarkers and 2 to sub-optimal biomarkers within the rule set (e.g., reflecting their relative clinical importance). By assigning different weights to abnormal and sub-optimal biomarkers, the system may better reflect the true impact of each biomarker on the health of the subject (e.g., at least with respect to that particular FBS subsystem). For example, with this approach, the system may distinguish between subjects who meet rule-set criteria with severely abnormal biomarkers from those with only mildly sub-optimal biomarkers. The denominator (3Zₘₐₓ) may represent the maximum possible weighted score if all biomarkers in the rule set were abnormal (i.e., 3×the total number of biomarkers). Table 3 illustrates an example weight mapping for this scenario (S+B+R).

In some examples, adopting a weighted approach for the Biomarker Rule Set Burden (R), similar to the one used for the Biomarker Burden Score (B), advantageously accounts for varying degrees of abnormality. For example, by using the biomarker rule set, the system recognizes that not all biomarkers may necessarily contribute equally to dysfunction. Furthermore, by weighting the biomarker rule set, the system may improve accuracy when estimating the probability of dysfunction while maintain consistency of its scoring methodology uniform across different scoring components.

TABLE 3
Example weight mapping for combined score based on S, B, and R

| | Symptom | Biomarker Weight | Biomarker Rule Set |
|---|---|---|---|
| FBS Subsystem | Weight (w_S) | (w_B) | Weight (w_R) |
| 1. Nutrition | 0.5 | 0.4 | 0.1 |
| 2. Lifestyle | 0.6 | 0.3 | 0.1 |
| 3. Medications | 0.4 | 0.5 | 0.1 |
| 4. Upper | 0.45 | 0.45 | 0.1 |
| Gastrointestinal | | | |
| System | | | |
| 5. Liver and | 0.25 | 0.6 | 0.15 |
| Gallbladder | | | |
| 6. Small Intestine | 0.45 | 0.45 | 0.1 |
| 7. Large Intestine | 0.45 | 0.45 | 0.1 |
| 8. Mineral Needs | 0.3 | 0.55 | 0.15 |
| 9. Essential Fatty | 0.35 | 0.5 | 0.15 |
| Acids | | | |
| 10. Blood Sugar | 0.25 | 0.6 | 0.15 |
| Regulation | | | |
| (Hyper) | | | |
| 11. Blood Sugar | 0.25 | 0.6 | 0.15 |
| Regulation | | | |
| (Hypo) | | | |
| 12. Vitamin Need | 0.4 | 0.5 | 0.1 |
| 13. Adrenal | 0.25 | 0.6 | 0.15 |
| (Hyper) | | | |
| 14. Adrenal | 0.25 | 0.6 | 0.15 |
| (Hypo) | | | |
| 15. Pituitary | 0.35 | 0.5 | 0.15 |
| (Hyper) | | | |
| 16. Pituitary | 0.35 | 0.5 | 0.15 |
| (Hypo) | | | |
| 17. Thyroid | 0.25 | 0.6 | 0.15 |
| (Hyper) | | | |
| 18. Thyroid | 0.25 | 0.6 | 0.15 |
| (Hypo) | | | |
| 19. Male Only | 0.35 | 0.5 | 0.15 |
| 20. Female Only | 0.35 | 0.5 | 0.15 |
| (Hyper) | | | |
| 21. Female Only | 0.35 | 0.5 | 0.15 |
| (Hypo) | | | |
| 22. | 0.25 | 0.6 | 0.15 |
| Cardiovascular | | | |
| 23. Kidney | 0.3 | 0.55 | 0.15 |
| 24. Bladder | 0.35 | 0.5 | 0.15 |
| 25. Immune | 0.4 | 0.4 | 0.2 |
| System | | | |
| 26. Cognitive | 0.4 | 0.45 | 0.15 |

In some examples, the system may assign different weights to different biomarkers in a biomarker rule set of a particular FBS subsystem (e.g., instead of or in addition to the weight w_{R}). For example, different biomarkers in the rule set may have varying degrees of clinical significance with respect to the potential functional condition. By weighting biomarkers within the rule set, for example, the system may capture the severity of deviations thereby providing a relatively more accurate reflection of the condition of the subject.

In some examples, the system may use a similar weight values (e.g., w_{B} and w_{R}) for both the general biomarker burden score (B) and the biomarker rule set burden score (R) to maintain consistency (e.g., so that it is easier for the user to interpret the scores and compare contributions of different components in the combined score (C)). In other examples, the system may assign different values for w_{B} and w_{R}.

In a particular embodiment, an example system may be configured to report (e.g., in a natural language format via a user interface of the system, etc.) an interpretation of the various burden scores (e.g., S, B, R, S+B, or S+B+R) according to a severity range that includes a particular burden score. For example, the system may interpret an optimal burden score (e.g., score within 0-10.99% range, etc.) as an assessment that minimal or no burden is detected for the particular FBS subsystem based on the relevant subject-reported symptom responses, biomarker lab results, etc., which may indicate a balanced functional state of this FBS subsystem. For example, an ‘optimal score’ may reflect a state (e.g., ideal state, etc.) where cellular processes are functioning as expected, with minimal symptoms, and/or within a healthy range. The system may also interpret a burden score within the mild range (e.g., 11-16.99%) as an assessment that a low burden is detected and/or minor imbalances detected. For example, a mild score level may be interpreted as presenting low or insignificant symptoms and/or can be used to indicate early signs of stress within the FBS subsystem (e.g., subject may benefit from preventative interventions). A score in the moderate range (e.g., 17-32.99%) may indicate a more pronounced imbalance with potential symptoms and/or lab deviations. For instance, at this level the FBS or subsystem may not function optimally, and/or the subject may begin experiencing noticeable symptoms indicating a need for targeted therapeutic support. For the high severity burden score range (e.g., more than 33%), the system may interpret the burden score as an assessment that a high burden (e.g., considerable dysfunction) is detected, which may correlate with pronounced symptoms and/or lab result abnormalities. For example, a high severity burden score may reflect a FBS or FBS subsystem that is under substantial stress, where intervention may facilitate restoring balance and/or support FBS function.

### Using Dynamically Computed and Weighted Scores to Predict Functional Conditions

In an embodiment, an example system is configured to predict functional conditions based on the combined score (‘C’). For example, the system may convert the combined score (i.e., severity score) into a probability value (e.g., between 0 and 1) that indicates the likelihood of dysfunction in a particular FBS subsystem. The following equation shows an example computation (e.g., logistic regression model) of the probability of dysfunction (‘P’) in a FBS subsystem based on C:

$$P = 1 / 1 + {e}^{- ( a + b C )}$$

In this example, e is Euler's number (e.g., ~2.71828), a is an intercept parameter (e.g., used to adjust the baseline probability), and b is a slope parameter (e.g., used to adjust sensitivity to changes in C). In some examples, the system is configured to set C to a value of 0 if the actual value of C is less than 0.1099.

The parameter a (e.g., intercept) may correspond to an intercept value of the logistic regression model. For example, adjusting parameter a may shift the logistic curve along the x-axis. A negative intercept parameter value (e.g., a=−1) may indicate to the example system that without any input (e.g., when the combined score C is zero, etc.) the baseline probability should be lower than 0.5, for example. The parameter b (e.g., slope parameter) may correspond to the slope of the logistic regression model. For example, parameter b may be adjusted to control the steepness of the logistic curve. For instance, a higher value of b may result in a steeper curve, i.e., small changes in C will be mapped to more significant changes in the predicted probability. For instance, in a scenario where b=6, the logistical curve may correspond to a relatively steep curve, i.e., the combined score strongly influences the computed probability to emphasize the importance of biomarkers in the combined probability for example.

In one embodiment, parameter ‘a’ may have a value of −1 and parameter ‘b’ may have a value of 6. However, other parameter values are possible as well. In one embodiment, an example system is configured to receive input from a user configured to set a user-defined value for parameters a, b, and/or any of the other parameters (e.g., weights, etc.) described above. In one embodiment, the example system is configured to use a machine learning model (e.g., neural network, large language model, etc.) trained using subject data (e.g., symptom data, biomarker data, and diagnosed functional conditions) to generate optimized values for the various parameters and weights described herein that improve the accuracy of the computed probability (P). Thus, some examples herein involve using artificial intelligence (e.g., machine learning) to automatically determine a combination of parameters for enhancing prediction accuracy of the computed probability (P).

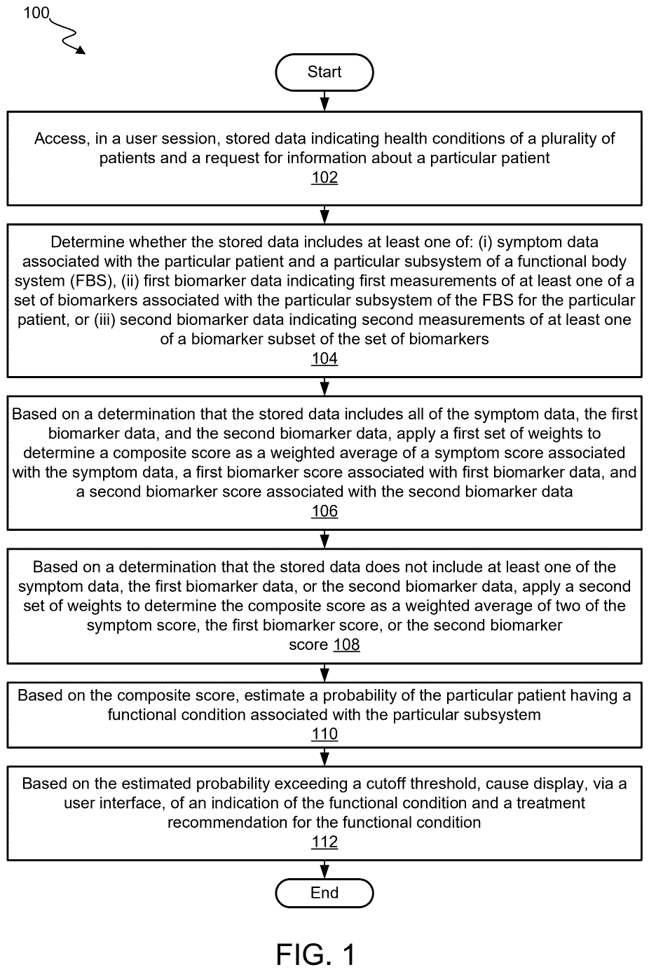

FIG. 1 illustrates a flow chart of an example process 100 that predicts body dysfunctions and their severity using various combinations of symptom data and/or biomarker data. At 102, the method 100 involves an example dysfunction prediction system accessing, in a user session, stored data indicating health conditions of a plurality of subjects and a request for information about a particular subject. At 104, the example dysfunction prediction system determines whether the stored data includes at least one of: (i) symptom data associated with the particular subject and a particular subsystem of a functional body system (FBS), (ii) first biomarker data indicating first measurements of at least one of a set of biomarkers associated with the particular subsystem of the FBS for the particular subject, or (iii) second biomarker data indicating second measurements of at least one of a biomarker subset of the set of biomarkers. At 106, the dysfunction prediction system applies a first set of weights to determine a composite score as a weighted average of a symptom score associated with the symptom data, a first biomarker score associated with first biomarker data, and a second biomarker score associated with the second biomarker data based on a determination that the stored data includes all of the symptom data, the first biomarker data, and the second biomarker data. At 108, the dysfunction prediction system applies a second set of weights to determine the composite score as a weighted average of one or more of the symptom score, the first biomarker score, or the second biomarker score based on a determination that the stored data does not include at least one of the symptom data, the first biomarker data, or the second biomarker data. At 110, the dysfunction prediction estimates a probability of the particular subject having a functional condition associated with the particular subsystem based on the composite score (e.g., ‘C’). At 112, the example dysfunction prediction system causes display, via a user interface, of an indication of the functional condition and a treatment recommendation for the functional condition based on the estimated probability exceeding a cutoff threshold.

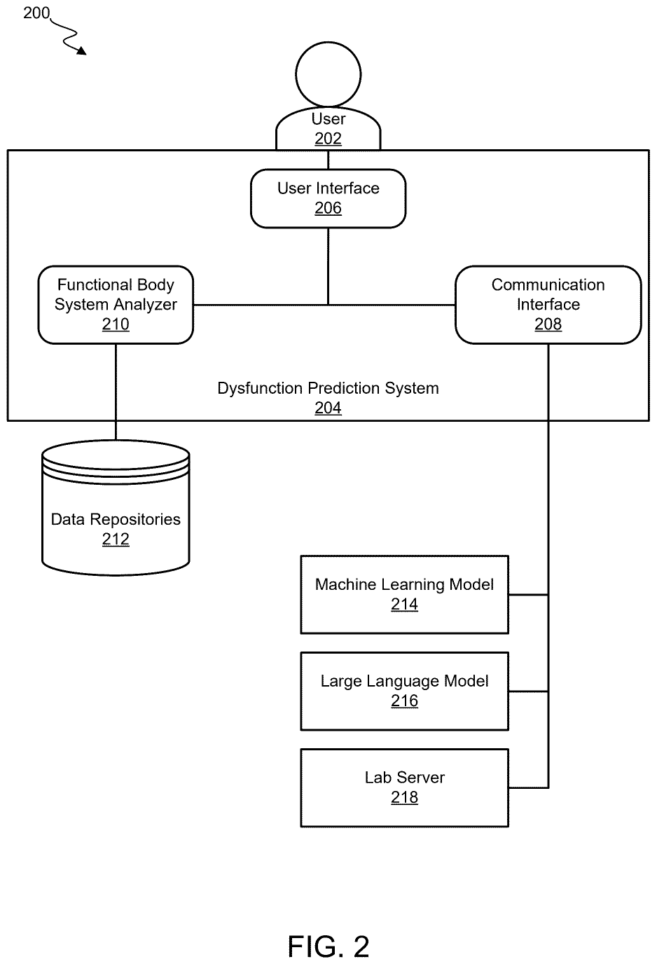

FIG. 2 illustrates a system diagram 200 showing an example dysfunction prediction system 204 that predicts body dysfunctions using a combination of symptom data and biomarker data. The dysfunction prediction system 204 includes a user interface 206 at which a user 202 (e.g., clinician) may request information about a particular subject. The dysfunction prediction system 204 also includes a communication interface 208 configured to communicate with various remote components or devices, such as machine learning model 214, large language model 216, and/or lab server 218. The dysfunction prediction system 204 also includes a FBS analyzer 210 configured to access stored data in the data repositories 212 and use the stored data to predict functional conditions associated with a particular FBS subsystem of a particular subject indicated by the user 202 via the user interface 206 (e.g., in a user session). To that end, the data repositories 212 may include one or more databases storing data such as symptom data, biomarker data, treatment data, and so on.

### Automated Generation of Personalized Clinical Evaluations, Treatment Plans, and Lab Testing Recommendations

In some embodiments, an example system is configured to display a severity flag (e.g., graphical indicator) indicating severity of the predicted dysfunction based on a combination of the computed symptom severity score (‘S’) and computed dysfunction probability (‘P’). Example severity flags may include a ‘Mild Dysfunction’ flag when a low to moderate probability (e.g., P less than 0.75) and a low symptom severity (e.g., S less than 17%) is computed, a ‘Moderate Dysfunction’ flag when a moderate to high probability (e.g., P in the range of 0.5 to 0.9) and a moderate symptom severity (e.g., S in the range of 17 to 33%) is computed, and a ‘Severe Dysfunction’ flag when a high to very high probability (e.g., P greater than 0.75) and a high symptom severity (e.g., S greater than 33%) is computed. It is noted that the various thresholds (e.g., 0.75, 17%, etc.) described above are only for the sake of example. Other thresholds are possible as well depending on various applications without departing from the scope of the present disclosure.

As an example, consider a scenario where a subject has a combined burden score (C) of 0.6 associated with a particular FBS subsystem. In this example, the system may use values of a=−1 and b=6 for the ‘a’ and ‘b’ parameters. Thus, the following equations can be solved to determine the probability of whether the subject has a dysfunction associated with the particular CBS subsystem.

$$P = 1 / 1 + e - ( - 1 + 6 \times 0 \text{.6} )$$

$$P = 1 / 1 + 2.7182 {8}^{⋀} - ( - 1 + 6 \times 0.6 ) = 1 / 1 + e - 2.61 \approx 0.931$$

In this example, the symptom severity percentage may be 35% and the probability that the subject has the dysfunction is relatively high (e.g., 93.1%). In this case, the example system may be configured to display (e.g., via the user interface) a severity flag (e.g., graphical indicator) corresponding to a severe dysfunction associated with that particular CBS subsystem.

As another example, the system may compute that the symptom severity percentage(S) of a subject for the Adrenal (Hyper) FBS subsystem is 30%, the biomarker burden score (B) is 47%, the biomarker rule set burden (R) is 60%, w_{S} of 0.25, w_{B} of 0.6, and w_{R} of 0.15. In this example, the system may compute the combined score (C) and the probability of dysfunction (P) as follows:

$$C = {w}_{S} ( {S}_{T} / M ) + {w}_{B} B + {w}_{R} R$$

$$C = 0. 25 \times 0.30 + 0.60 \times 0.47 + 0.15 \times 0.60$$

$$C = 0. 075 + 0.282 + 0 \text{.09} C = 0.4 \; 47$$

$$P = 1 / 1 + e - ( a + b C )$$

$$P = 1 / 1 + e - ( - 1 + 6 \times 0.447 )$$

$$P = 1 / 1 + e - ( - 1 + 2.682 )$$

$$P = 1 / 1 + e - 1682$$

$$P = 1 / 1 + 0.186$$

$$P = 1 / 1.186$$

$$P \approx 0. 843$$

In this example, the probability (P) is approximately 84.3% and the system severity is approximately 30% (e.g., Moderate). Thus, in this example, the system may be configured to display a severity flag or natural language output corresponding to “Moderate to Severe Adrenal Hyperfunction,” since the probability is high (e.g., greater than 80%) and the symptom severity is moderate.

In some embodiments, an example system is configured to display an interpretation of the computed probability of dysfunction (e.g., in the form of a natural language output via a user interface of the system, etc.). In this example, the system may determine the output by comparing the computed probability (P) with one or more ranges of probability values (e.g., cutoffs). For example, the system may use a mapping of cutoff ranges to categorize the severity of a potential disease or dysfunction state of the subject. Such cutoffs may advantageously assist a user (e.g., clinicians) in making informed decisions and/or communicating effectively with subjects.

By way of example, if P is less than 0.4 (i.e., less than 40%), the system may interpret the result as a low probability (e.g., indicated using a green colored indicator or other graphical indicator in the user interface). If P is between 0.401 and 0.75 (i.e., range of 40.01% to 75%), the system may interpret the probability as a moderate probability (e.g., indicated by a yellow color indicator, etc.). If P is between 0.7501 and 0.9 (i.e., range of 75.01% to 90%), the system may interpret P as a high probability (e.g., indicated using an orange graphical indicator). And if P is higher than 0.901 (e.g., 90.01% and above), then the system may interpret the computed P as being a very high probability (e.g., red color graphical indicator, etc.).

For example, a ‘low probability’ (e.g., P less than 0.4) interpretation may indicate to the user that there is a less than 40% estimated chance of dysfunction. In this example, the system may display to the user a recommendation indicating that no immediate intervention specific to this dysfunction is recommended, a recommendation that the user should continue monitoring the subject, and/or a recommendation that the user consider other possible causes of symptoms. For instance, probabilities below 50% may suggest that a dysfunction is less likely and/or that other factors may contribute to the condition (e.g., symptoms, etc.) of the subject.

As another example, a ‘moderate probability’ (e.g., P in the range of 0.4001 to 0.75, etc.) interpretation may indicate to the user that there is a 40 to 75% estimated chance of dysfunction. In this example, the system may display to the user a recommendation to consider further diagnostic evaluations, monitor symptoms closely, and/or initiate mild interventions or lifestyle modifications. For instance, probabilities in this range may indicate a moderate likelihood of dysfunction which may warrant cautious attention without necessarily immediate aggressive treatment.

As another example, a ‘high probability’ (e.g., P in the range of 0.75 to 0.9, etc.) interpretation may indicate to the user that there is a 75 to 90% estimated chance of dysfunction. In this example, the system may display to the user a recommendation to initiate appropriate interventions such as targeted therapies, and/or monitor subject response closely. For instance, a high probability may suggest dysfunction is likely present and thus proactive management is recommended.

As another example, a ‘very high probability’ (e.g., P greater than 0.9001, etc.) interpretation may indicate to the user that there is a 90% or higher estimated chance of dysfunction. In this example, the system may display to the user a recommendation to implement immediate or more aggressive interventions, conduct comprehensive evaluations, and/or consider referrals to specialists. For instance, probabilities at or above 90% may suggest a high likelihood of dysfunction and thus prompt/decisive clinical action is recommended.

In some examples, the various probability cutoff values (e.g., 0.5, 0.75, 0.9, etc.) used to categorize the computed probability (P) may be determined using a machine learning model. By way of example, the system may calibrate the model (i.e., including the cutoff values and other parameters in the equations of P and C) using data from a specific subject population of a user (e.g., clinician) to refine the cutoff points for that specific user. For example, the system may train a machine learning (ML) model and/or configure a large language model (LLM) using the subject data of the user so that the ML model or LLM can predict a combination of cutoff values that are likely to provide a more suitable interpretation of the computed probability with respect to the subject population of that particular user.

Other probability ranges and/or cutoff values are possible as well. In some examples, the probability cutoff values can be user-defined (e.g., input by the user via the user interface). In some examples, the system may be configured to review and/or adjust the probability cutoff values based on emerging evidence and/or clinical outcomes of subject treatment. For instance, the system may align the cutoffs with common clinical decision thresholds, e.g., to balance sensitivity with specificity and/or to differentiate between levels of urgency in clinical response.

In some scenarios, subjects may have unique factors affecting their risk. Accordingly, in some embodiments, an example system is configured to provide its interpretation to the user via the user interface as a draft interpretation, which the user can adjust (e.g., via the user interface) before sharing with the subject or confirming for storage in the subjects record at the system. Thus, in some scenarios cutoffs at 40%, 75%, and 90% may be intuitive breaking points in probability, e.g., corresponding to increasing levels of certainty. Whereas, in other scenarios, different cutoff values or thresholds may be more suitable. In general, cutoff thresholds may be selected with the aim of minimizing false negatives (e.g., missing a dysfunction) while keeping false positives (e.g., over diagnosing) at an acceptable level. In some examples, the system may be configured to provide an indication of the cutoff thresholds currently applied (e.g., clear categories such as low probability, moderate probability, etc.) to the user so as to assist in explaining the likelihood of dysfunction to subjects and thus aid in shared decision making.

In some embodiments, an example system is configured to suggest specific laboratory tests to assess various FBS subsystems based on the computed probability (P) and/or combined score (C). Upon receiving lab results (e.g., lab user submits results to system via an API, or clinician user inputs lab results to system), the system may evaluate and/or and classifies the lab results to update the FBS Burden Scores (e.g., B, R, C, etc.) of relevant FBS subsystems and/or update the probability of dysfunction (P), which may also be referred to herein as the Predictive Dysfunction Index (PDI).

In general, P or PDI is a metric that assesses the likelihood of specific functional conditions in subjects. By analyzing a combination of subject-reported symptoms, biomarkers associated with Functional Body Subsystems (FBS), and established biomarker rule sets, the PDI generates a probability score indicating the potential presence of various functional conditions. In line with the discussion above, a functional condition refers to a dysfunction (e.g., which may be described using conventional medical terminology).

In some embodiments, an example system is configured to receive (e.g., via a user interface) input from the user (e.g., clinician) that selects whether the user wants to promote a PDI or a dysfunction prediction to be listed in the user interface as a functional condition (e.g., thereby confirming the diagnosis of the predicted dysfunction and signifying intent to treat). In this example, the system may be configured to highlight the dysfunction on a Subject Summary section of the user interface.

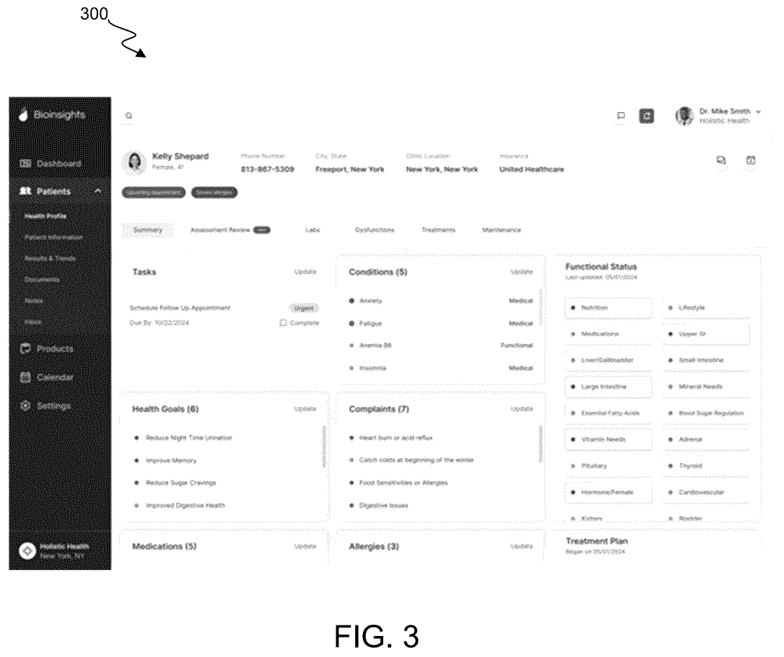

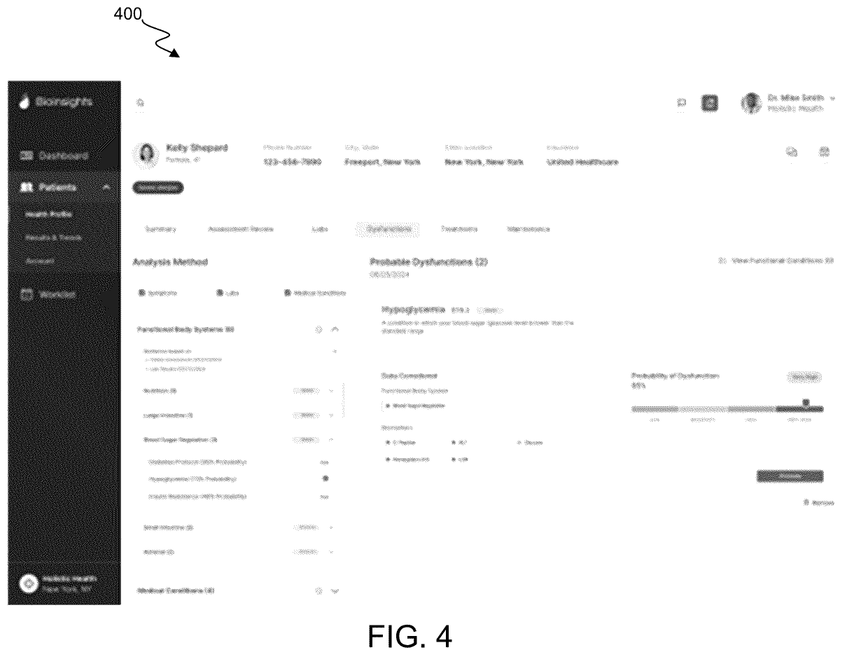

FIG. 3 illustrates a diagram of an example user interface 300 showing an example subject profile summary. FIG. 4 illustrates a diagram of an example user interface 400 showing an example dysfunction prediction.

In some embodiments, an example system includes a treatment repository or database that stores a plurality of possible treatments associated with one or more respective functional conditions. For example, the system may generate an output (e.g., via the user interface) that recommends one or more treatments associated with a functional condition of a particular subject of the user (e.g., based on the functional condition being flagged as sever or promoted by the user).

In some embodiments, when biomarker data is unavailable for a subject, the system applies a symptom-only evaluation method. For example, the system may determine the symptom score based on symptom data and determine the composite score based on the symptom score (e.g., by applying a weight of 1 to the symptom score). Additionally or alternatively, the system may evaluate symptom data against one or more trigger rules or threshold conditions, and, based on a determination that a trigger rule is satisfied, cause display (e.g., via the user interface) of an indication (e.g., a link) of a corresponding functional condition and output a treatment recommendation from the treatment repository without requiring biomarker data.

### Computer System Architecture

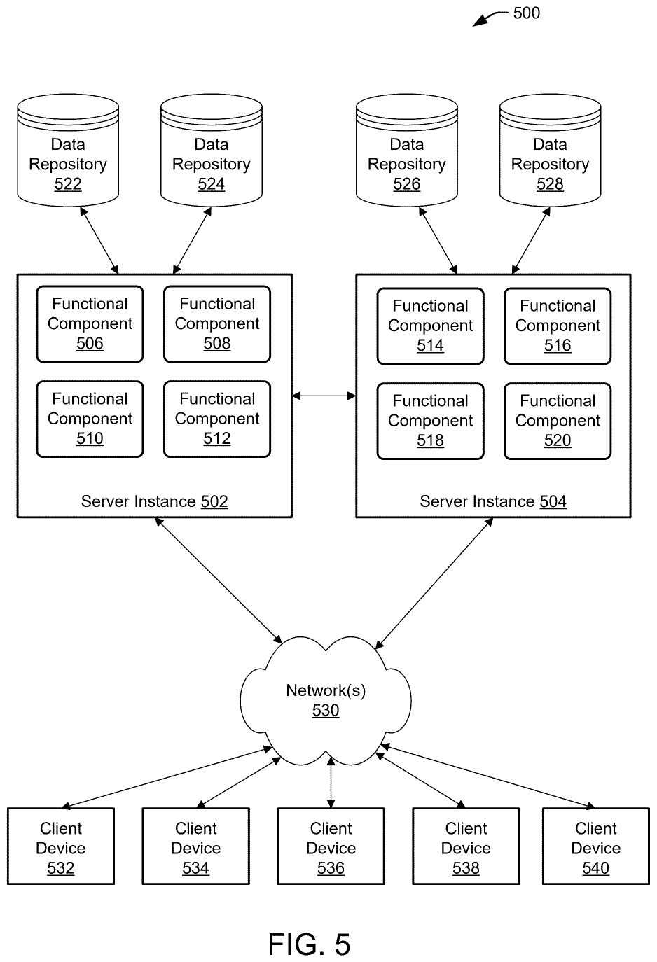

FIG. 5 illustrates a diagram of a distributed system 500 for functional body system assessment and dysfunction prediction using symptom and biomarker data.

The example system 500 includes client computing devices 532-540 in communication with a server 502, 504 via one or more communication networks 530. Users may use the client computing devices 532-540 to access services offered by the server 502, 504. The client computing devices 532-540 may include smart phones or other portable handheld devices, general purpose computers such as personal computers and laptops, workstation computers, personal assistant devices, wearable devices, equipment firmware, gaming systems, and the like.

The distributed system 500 provides services for predicting body dysfunctions using a combination of subjective symptom data and objective biomarker data. The server 502, 504 includes one or more components 506-520 that are configured to perform functions to carry out the services.

These client and server devices may use various operating systems, such as any of Microsoft Windows®, Apple MacOS®, UNIX® or UNIX-like operating systems, Linux® or Linux-like operating systems, Android®, and Apple iOS®, which provide software access to underlying hardware resources.

The client and server computing devices may include one or more data repositories 522-528 to store data, including instructions and data or other stored objects on which the instructions operate. The client and server devices of the system 500 may include one or more data processor and one or more non-transitory computer-readable storage media storing instructions which, when executed on the one or more data processors, cause the one or more data processors to perform part or all of one or more methods disclosed herein. The system 500 may also include one or more computer-program products tangibly embodied in a non-transitory machine-readable storage medium that stores instructions configured to cause one or more data processors to perform part or all of one or more methods disclosed herein. In various embodiments, part or all of one or more methods disclosed herein may be performed by stored instructions execution on one or more of client devices 532-540 and/or servers 502-504.

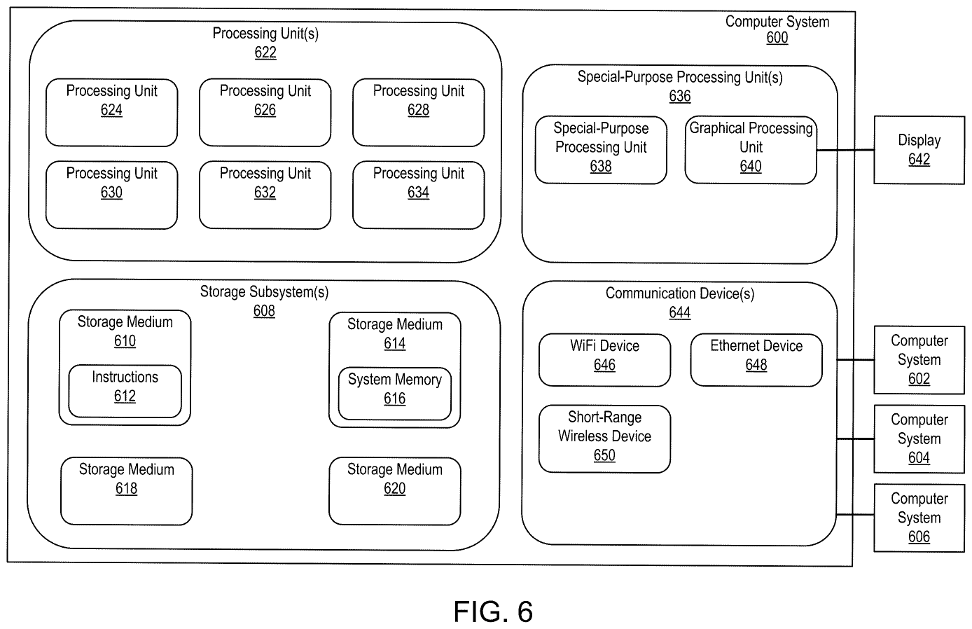

FIG. 6 illustrates an example computer system 600 that may be used to implement certain aspects. As shown in FIG. 6, computer system 600 includes various subsystems including a processing subsystems 622 and 636, a storage subsystem 608, and a communications subsystem 644. Processing subsystems 622, 636 may include one or more processors 624-634 and 638-640 configured to execute instructions 612 to cause the system 600 to perform the functions of the methods of the present disclosure. Storage subsystem 644 may include non-transitory computer-readable storage media 610, 614, 618, 620 including storage media 614 and a system memory 616. Computer system 600 may also include input devices such as mice, keyboards, touchscreens, touchpads, buttons, joysticks, and output devices such as a display and speaker. For example, the computer system 600 may operate the display 642 using the graphical processing unit 640. Computer system 600 may also include one or more communication devices 644, such as WiFi Device 646, ethernet device 648, and/or short range wireless device 640. The computer system 600 may use the communication devices 644 to communicate with one or more other computer systems 602, 604, 606.

Storage subsystem may include one or more non-transitory memory devices, including volatile and non-volatile memory devices. As shown, storage subsystem 608 includes a system memory 616 and a computer-readable storage media 610, 614, 618, 620. System memory 616 may include a number of memories including a volatile main random access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system, such as during start-up, may typically be stored in the ROM. The RAM typically contains data and/or program modules in the process of being operated and executed by processing subsystem. In some implementations, system memory may include multiple different types of memory, such as static random access memory (SRAM), dynamic random access memory (DRAM), and the like.

Computer-readable media 610 may store computer-readable instructions 612, data structures, program modules, and other data for computer system to operate. Software (programs, code modules, instructions) that, when executed by processing subsystem provides the functionality described above, may be stored in storage subsystem. By way of example, computer-readable storage media may include non-volatile memory such as a hard disk drive, a magnetic disk drive, an optical disk drive such as a CD ROM, digital video disc (DVD), a Blu-Ray® disk, or other optical media, flash memory cards, universal serial bus (USB) flash drives, secure digital (SD) cards. Computer-readable media may also include solid-state drives (SSD) based on volatile or non-volatile memory.

Communication subsystem 644 may support both wired (e.g., Ethernet) and/or wireless communication protocols (e.g., WiFi, Bluetooth, etc.).

Although specific aspects have been described, various modifications, alterations, alternative constructions, and equivalents are possible. Embodiments are not restricted to operation within certain specific data processing environments, but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claimed is:

1. A computer-implemented method comprising:
accessing, in a user session, stored data indicating health conditions of a plurality of subjects and a request for information about a particular subject;
determining whether the stored data includes at least one of: (i) symptom data associated with the particular subject and a particular subsystem of a functional body system (FBS), (ii) first biomarker data indicating first measurements of at least one of a set of biomarkers associated with the particular subsystem of the FBS for the particular subject, or (iii) second biomarker data indicating second measurements of at least one of a biomarker subset of the set of biomarkers, the biomarker subset being identified in a biomarker rule set associated with the particular subsystem; wherein the symptom data indicates one or more symptoms associated with the particular subsystem and reported by the subject;
based on a determination that the stored data includes all of the symptom data, the first biomarker data, and the second biomarker data, applying a first set of weights to determine a composite score as a weighted average of a symptom score associated with the symptom data, a first biomarker score associated with first biomarker data, and a second biomarker score associated with the second biomarker data;
based on a determination that the stored data does not include at least one of the symptom data, the first biomarker data, or the second biomarker data, applying a second set of weights to determine the composite score as a weighted average of one or more of the symptom score, the first biomarker score, or the second biomarker score;
based on the composite score, estimating a probability of the particular subject having a functional condition associated with the particular subsystem; and
based on the estimated probability exceeding a cutoff threshold, causing display, at a user interface, of an indication of the functional condition and a treatment recommendation for the functional condition.

2. The computer-implemented method of claim 1, further comprising:
based on a determination that the stored data includes the symptom data of the particular subject, determining the symptom score of the particular subject for the particular subsystem of the FBS based on the symptom data, wherein the symptom score indicates symptom severity of the one or more symptoms reported by the subject relative to a maximum symptom severity associated with the particular subsystem.

3. The computer-implemented method of claim 1, further comprising:
based on a determination that the stored data includes the first biomarker data, determining the first biomarker score of the particular subject for the particular subsystem of the FBS based on the first biomarker data, wherein the first biomarker score characterizes biomarker abnormality indicated by the first measurements relative to a maximum biomarker abnormality associated with the set of one or more biomarkers of the particular subsystem.

4. The computer-implemented method of claim 1, further comprising:
based on a determination that the stored data includes the second biomarker data, determining the second biomarker score of the particular subject for the particular subsystem of the FBS based on the second biomarker data, wherein the second biomarker score characterizes biomarker abnormality indicated by the second measurements relative to a maximum biomarker abnormality associated with the biomarker subset of the biomarker rule set.

5. A computer-program product comprising one or more non-transitory machine-readable storage media, including stored instructions configured to cause a computing system to perform a set of actions including:
accessing, in a user session, stored data indicating health conditions of a plurality of subjects and a request for information about a particular subject;
determining whether the stored data includes at least one of: (i) symptom data associated with the particular subject and a particular subsystem of a functional body system (FBS), (ii) first biomarker data indicating first measurements of at least one of a set of biomarkers associated with the particular subsystem of the FBS for the particular subject, or (iii) second biomarker data indicating second measurements of at least one of a biomarker subset of the set of biomarkers, the biomarker subset being identified in a biomarker rule set associated with the particular subsystem; wherein the symptom data indicates one or more symptoms associated with the particular subsystem and reported by the subject;
based on a determination that the stored data includes all of the symptom data, the first biomarker data, and the second biomarker data, applying a first set of weights to determine a composite score as a weighted average of a symptom score associated with the symptom data, a first biomarker score associated with first biomarker data, and a second biomarker score associated with the second biomarker data;
based on a determination that the stored data does not include at least one of the symptom data, the first biomarker data, or the second biomarker data, applying a second set of weights to determine the composite score as a weighted average of one or more of the symptom score, the first biomarker score, or the second biomarker score;
based on the composite score, estimating a probability of the particular subject having a functional condition associated with the particular subsystem; and
based on the estimated probability exceeding a cutoff threshold, causing display, at a user interface, of an indication of the functional condition and a treatment recommendation for the functional condition.

6. The computer-program product of claim 5, wherein the set of actions further includes;
based on a determination that the stored data includes the symptom data of the particular subject, determining the symptom score of the particular subject for the particular subsystem of the FBS based on the symptom data, wherein the symptom score indicates symptom severity of the one or more symptoms reported by the subject relative to a maximum symptom severity associated with the particular subsystem.

7. The computer-program product of claim 5, wherein the set of actions further includes:
based on a determination that the stored data includes the first biomarker data, determining the first biomarker score of the particular subject for the particular subsystem of the FBS based on the first biomarker data, wherein the first biomarker score characterizes biomarker abnormality indicated by the first measurements relative to a maximum biomarker abnormality associated with the set of one or more biomarkers of the particular subsystem.

8. The computer-program product of claim 5, wherein the set of actions further includes:
based on a determination that the stored data includes the second biomarker data, determining the second biomarker score of the particular subject for the particular subsystem of the FBS based on the second biomarker data, wherein the second biomarker score characterizes biomarker abnormality indicated by the second measurements relative to a maximum biomarker abnormality associated with the biomarker subset of the biomarker rule set.

9. A system comprising:
one or more processors; and
one or more non-transitory computer-readable media storing instructions, which, when executed by the system, cause the system to perform a set of actions including:
accessing, in a user session, stored data indicating health conditions of a plurality of subjects and a request for information about a particular subject;
determining whether the stored data includes at least one of: (i) symptom data associated with the particular subject and a particular subsystem of a functional body system (FBS), (ii) first biomarker data indicating first measurements of at least one of a set of biomarkers associated with the particular subsystem of the FBS for the particular subject, or (iii) second biomarker data indicating second measurements of at least one of a biomarker subset of the set of biomarkers, the biomarker subset being identified in a biomarker rule set associated with the particular subsystem; wherein the symptom data indicates one or more symptoms associated with the particular subsystem and reported by the subject;
based on a determination that the stored data includes all of the symptom data, the first biomarker data, and the second biomarker data, applying a first set of weights to determine a composite score as a weighted average of a symptom score associated with the symptom data, a first biomarker score associated with first biomarker data, and a second biomarker score associated with the second biomarker data;
based on a determination that the stored data does not include at least one of the symptom data, the first biomarker data, or the second biomarker data, applying a second set of weights to determine the composite score as a weighted average of one or more of the symptom score, the first biomarker score, or the second biomarker score;
based on the composite score, estimating a probability of the particular subject having a functional condition associated with the particular subsystem; and
based on the estimated probability exceeding a cutoff threshold, causing display, at a user interface, of an indication of the functional condition and a treatment recommendation for the functional condition.

10. The system of claim 9, wherein the set of actions further includes;
based on a determination that the stored data includes the symptom data of the particular subject, determining the symptom score of the particular subject for the particular subsystem of the FBS based on the symptom data, wherein the symptom score indicates symptom severity of the one or more symptoms reported by the subject relative to a maximum symptom severity associated with the particular subsystem.

11. The system of claim 9, wherein the set of actions further includes:
based on a determination that the stored data includes the first biomarker data, determining the first biomarker score of the particular subject for the particular subsystem of the FBS based on the first biomarker data, wherein the first biomarker score characterizes biomarker abnormality indicated by the first measurements relative to a maximum biomarker abnormality associated with the set of one or more biomarkers of the particular subsystem.

12. The system of claim 9, wherein the set of actions further includes:
based on a determination that the stored data includes the second biomarker data, determining the second biomarker score of the particular subject for the particular subsystem of the FBS based on the second biomarker data, wherein the second biomarker score characterizes biomarker abnormality indicated by the second measurements relative to a maximum biomarker abnormality associated with the biomarker subset of the biomarker rule set.
